(19) United States
(12) Patent Application Publication
(10) Pub. No.: US 2020/0359979 A1
(43) Pub. Date: Nov. 19, 2020

(54) ARTIFICIAL INTELLIGENCE BASED AUTOMATIC MARKER PLACEMENT IN RADIOGRAPHIC IMAGES

(71) Applicant: CARESTREAM HEALTH, INC., Rochester, NY (US)

(72) Inventors: Xiaohui WANG, Pittsford, NY (US); Martin S. PESCE, Quakertown, PA (US)

(21) Appl. No.: 16/869,631

(22) Filed: May 8, 2020

Related U.S. Application Data
(60) Provisional application No. 62/846,790, filed on May 13, 2019.

(51) Int. Cl. A61B 6/00

(52) U.S. Cl. CPC A61B 6/469; A61B 6/542; A61B 6/463

(57) ABSTRACT

A method of operating a digital radiographic imaging system includes capturing a digital radiographic image of a subject anatomy and automatically placing a digital anatomical marker therein. The radiographic image is displayed to an operator, the operator confirming a position of the automatically placed digital anatomical marker by storing the radiographic image or by repositioning the marker and storing the radiographic image.

### CROSS REFERENCE TO RELATED APPLICATIONS

This application claims priority to U.S. Patent Application Ser. No. 62/846,790, filed May 13, 2019, in the name of Wang, et al., and entitled AI-BASED AUTOMATIC MARKER PLACEMENT IN RADIOGRAPHIC IMAGES, which is hereby incorporated by reference herein in its entirety.

### BACKGROUND OF THE INVENTION

The disclosure relates generally to the field of radiography and in particular to operator utilities for workflow support. More specifically, the disclosure relates to methods and systems using artificial intelligence (AI) for automated anatomical marker placement in the digital radiographic image.

As a standard practice in most radiographic facilities, radiography personnel position radio-opaque anatomical orientation markers within the image field of each x-ray image. Markers provide a straightforward tool for helping to distinguish left and right sides and/or front and back views of the imaged anatomy. Proper practice in applying radiographic markers helps to assure correct image orientation and thus to prevent misinterpretation in the diagnostic assessment of the acquired images.

Anatomical markers can provide radio-opaque letters or symbols that indicate left (L) and right (R) sides of the imaged patient anatomy or show other orientation data. Markers are typically small and placed unobtrusively, but are readily detectable within the processed image, and can have any suitable shape that allows them to be unambiguously visually perceptible. In some cases, where particular anatomy within the captured region may not be sufficiently distinctive, anatomical markers may indicate reference points for image interpretation. Often, markers can include other identifying information in addition to anatomy orientation, such as name, initials, or number assigned to the radiographic practitioner, the patient, or the facility, for example.

The conventional radio-opaque anatomical marker used for radiographic imaging onto x-ray film is formed from lead or other suitable radio-opaque material and can be clipped directly or coupled in some way onto the X-ray cassette. In typical practice, radiography technologists were often provided with a set of individualized markers for application as a part of standard setup and imaging workflow.

With the onset of digital radiography (DR) imaging systems, including both direct digital radiography (DR) and computed radiography (CR) systems, interactive electronic placement of anatomical markers is now practiced at many sites, with markers interactively positioned on-screen in the acquired image as a part of post-processing workflow, according to operator input. This practice changes the standard paradigm for marker use and offers some advantages, such as eliminating a possible source of infection, providing readily interpreted information, and reducing exposure to lead and overall lead use. However, on-screen anatomical marker placement and annotation can result in added steps to the operator workflow for post-processing and managing image content. On-screen marker manipulation can introduce possible sources of error, such as incorrect determination of anatomy or incorrect orientation, misplacement of markers within the image, or inadvertent omission of the marking step altogether. In placing markers on the image, the technologist or other operator of the digital system must be particularly alert for special conditions in which internal organs may be differently positioned than in the general case, such as in instances of situs inversus and dextrocardia.

One goal for improving workflow and efficiency of the radiography process is to reduce the number of operator steps needed to post-process the acquired DR image. Manual steps needed to determine anatomy orientation within an image, to specify a suitable anatomical marker type for the given anatomy, and to position the marker in the desired location on the acquired image can be time-consuming and may be prone to operator error.

Thus, it can be appreciated that there would be benefits to solutions that reduce the number of operator steps and decisions for positioning anatomical markers on acquired radiography images and thus simplify the task of on-screen digital marker placement within the digital radiography (DR) or computed radiography (CR) image.

The discussion above is merely provided for general background information and is not intended to be used as an aid in determining the scope of the claimed subject matter.

### BRIEF DESCRIPTION OF THE INVENTION

A method of operating a digital radiographic imaging system includes capturing a digital radiographic image of a subject anatomy and automatically placing a digital anatomical marker therein. The radiographic image is displayed to an operator, the operator confirming a position of the automatically placed digital anatomical marker by repositioning the marker or by not repositioning the marker. The captured radiographic image with the operator confirmed position of the digital anatomical marker is stored.

In one embodiment, a method for processing a digital radiographic image of a patient anatomy is disclosed. The method includes acquiring a digital radiographic image of the patient anatomy and automatically superimposing an anatomical marker at a first position within the image of the patient anatomy. A trained neural network determines a position of the anatomical marker after training using a plurality of digital radiographic images having anatomical markers placed therein. The anatomical marker indicates at least a left side or right side orientation of the patient anatomy within the digital radiographic image. A viewer of the displayed digital radiographic image indicates a preferred position of the anatomical marker by electronically storing the digital radiographic image with the anatomical marker at the first position or by repositioning the anatomical marker to a different position and then electronically storing the digital radiographic image.

In one embodiment, a method for processing a digital radiographic (DR) image executed at least in part by a computer is disclosed. The method includes acquiring a digital radiographic image of a patient anatomy having data identifying a region of interest (ROI) within the patient anatomy. The digital radiographic image of the patient anatomy is displayed with an anatomical marker superimposed by the computer at a computer determined first position outside the ROI. An operator instruction moves the anatomical marker from the first position to a second position offset from the first position, and the digital radiographic image is stored with the anatomical marker at the second position.

An object of the present disclosure is to advance the art of digital radiography and address the need for improved marker placement techniques within the acquired radiographic image.

Another object of the present disclosure is to provide automated utilities for marker placement that take advantage of standard imaging practices and help to remind the operator to complete and validate the image marking step.

These objects are given only by way of illustrative example, and such objects may be exemplary of one or more embodiments of the invention. There may be other desirable objectives and advantages apparent to those skilled in the art. The invention is defined by the appended claims.

The summary descriptions above are not meant to describe individual separate embodiments whose elements are not interchangeable. In fact, many of the elements described as related to a particular embodiment can be used together with, and possibly interchanged with, elements of other described embodiments. Many changes and modifications may be made within the scope of the present invention without departing from the spirit thereof, and the invention includes all such modifications.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings below are intended to be drawn neither to any precise scale with respect to relative size, angular relationship, relative position, or timing relationship, nor to any combinational relationship with respect to interchangeability, substitution, or representation of a required implementation, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1 is a schematic block diagram that shows a radiography system that uses a DR detector for image acquisition.

FIG. 2 shows a portion of a chest x-ray view having an anatomical marker applied using a digital radiography system.

FIG. 3 shows a view of left and right hands having markers applied using a digital radiography system.

FIG. 4 is a workflow sequence that shows a procedure for automating marker placement on the acquired image, according to an embodiment of the present disclosure.

FIGS. 5A and 5B are schematic diagrams that show aspects of image content that affect marker placement.

### DETAILED DESCRIPTION OF THE EMBODIMENTS

In the image processing context of the present disclosure, “rendering” is the active process of generating and forming a digital image for display and generating the pattern of signals needed for displaying the image to a user. An image file contains objects in a strictly defined language or data structure, defining aspects of the image content such as geometry, viewpoint, texture, lighting, and shading information. The data contained in the file is passed to a rendering program to be processed and output or streamed to a display driver or graphics processing unit (GPU) for direct presentation on a display or to a digital image or raster graphics image file. The same image content can be rendered, for example, on a monochrome display or in color on a full color display.

With respect to an image detector, the term “pixel” refers to a picture element unit cell containing a photo-conversion circuit and related circuitry for converting incident electromagnetic radiation to an electrical signal. For the image processing steps described herein, the terms “pixels” for picture image data elements, conventionally used with respect to 2-D imaging and image display, and “voxels” for volume image data elements, often used with respect to 3-D imaging, can be used interchangeably. It should be noted that the 3-D volume image is itself synthesized from image data obtained as pixels on a 2-D sensor array and displays as a 2-D image from some angle of view. Thus, 2-D image processing and image analysis techniques can be applied to the 3-D volume image data.

The term “highlighting” for a displayed feature has its conventional meaning as is understood to those skilled in the information and image display arts. In general, highlighting uses some form of localized display enhancement to attract the attention of the viewer. Highlighting a portion of an image, such as a marker or an individual organ, bone, or structure, or a path from one chamber to the next, for example, can be achieved in any of a number of ways, including, but not limited to, annotating, displaying a nearby or overlaying symbol, outlining or tracing, display in a different color or at a markedly different intensity or gray scale value than other image or information content, blinking or animation of a portion of a display, or display at higher sharpness or contrast.

In the context of the present disclosure, the general term “digital radiography” or “DR” is considered to encompass both direct digital radiography (DR) that is electronically obtained from a two-dimensional array of photosensors that is placed in the path of ionizing radiation, and computed radiography (CR) that temporarily stores exposure data on a phosphor plate or sheet that is subsequently scanned in order to extract the radiographic image content. In both technologies, digital data is generated from an x-ray exposure, with the data stored as a file or streamed as data to a display or storage device.

The term “in signal communication” as used in the application means that two or more devices and/or components are capable of communicating with each other via signals that travel over some type of signal path. Signal communication may be wired or wireless. The signals may be communication, power, data, or energy signals which may communicate information, power, and/or energy from a first device and/or component to a second device and/or component along a signal path between the first device and/or component and second device and/or component. The signal paths may include physical, electrical, magnetic, electromagnetic, optical, wired, and/or wireless connections between the first device and/or component and second device and/or component. The signal paths may also include additional devices and/or components between the first device and/or component and second device and/or component.

The term “set”, as used herein, refers to a non-empty set, as the concept of a collection of elements or members of a set is widely understood in elementary mathematics. The term “subset”, unless otherwise explicitly stated, is used herein to refer to a non-empty proper subset, that is, to a subset of the larger set, having one or more members. For a set S, a subset may comprise the complete set S. A “proper subset” of set S, however, is strictly contained in set S and excludes at least one member of set S.

In the context of the present disclosure, the terms “marker”, “radiographic marker”, “anatomical marker”, and “orientation marker” are used synonymously, except where otherwise indicated, and refer to a symbol or symbols, added to the original radiographic image, that indicates the spatial orientation of the imaged anatomy or, alternately considered, indicating the view orientation as rendered. Following conventional practice, anatomical marker content may be indicative of at least the front- or rear-view orientation of the patient anatomy within a particular rendered image. It can be readily appreciated that this front- or rear-view indication may be equivalent to identifying right (R) or left (L) side orientation of the anatomy as the anatomy appears in the rendered image. Typically, the L and R symbols are inverted when the acquired image is viewed from the rear, relative to its acquired position.

In the context of the present disclosure, the terms “neural network logic” and “machine-learning logic” are used equivalently; neural network logic is trained or conditioned using a training set containing a large number of example x-ray images.

In typical applications, a computer or other type of dedicated logic processor for obtaining, processing, and storing image data is part of the 2-D radiography system, along with one or more displays for viewing image results. A computer-accessible memory is also provided, which may be a memory storage device used for longer term storage, such as a device using magnetic, optical, or other data storage media. In addition, the computer-accessible memory can comprise an electronic memory such as a random-access memory (RAM) that is used for shorter term storage, such as employed to store a computer program having instructions for controlling one or more computers to practice the method according to the present invention.

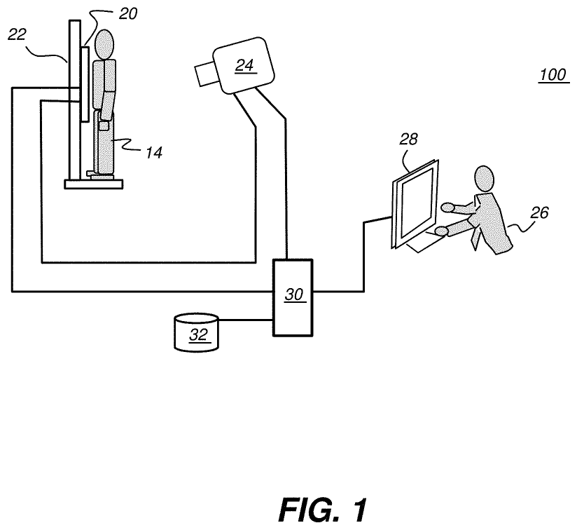

The schematic block diagram of FIG. 1 shows a radiography system 100 that uses a DR detector 20. A patient 14 is positioned adjacent DR detector 20 mounted on a bucky or other support 22, and exposed to radiation from an x-ray source 24 for image capture. Following x-ray image capture, the technologist or other practitioner 26 can view the captured image on a display console of a workstation 28.

Practitioner 26 controls setup and operation from workstation control console or display 28. A control logic processor 30 is in signal communication with other components of radiography system 100 and provides control signals for the x-ray source 24 generator and for data acquisition from detector 20. A memory 32 or other storage apparatus, in signal communication with control logic processor 30, can store the acquired image data.

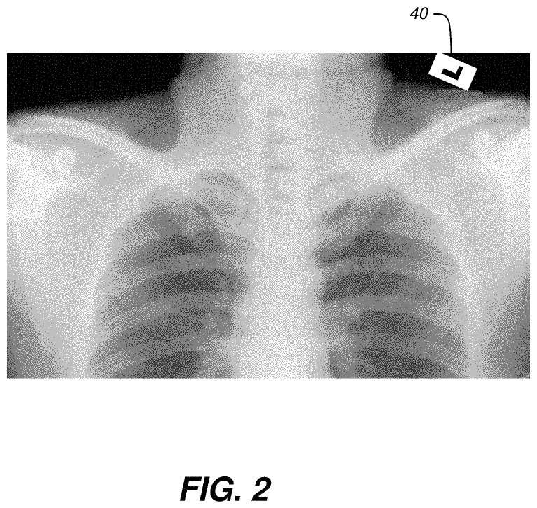

The plan view of FIG. 2 shows a portion of a chest x-ray view having orientation or anatomical marker 40 added during post-processing of the image. Marker 40 can be positioned on the acquired digital x-ray image using workstation display 28 of FIG. 1. In this example, the anatomical marker 40 indicates the left side (L) of the patient by being appropriately positioned to one side (or the other) of the spine or central area of the image. This marker 40 placement can help to identify specific features in the image and to prevent confusion in distinguishing the front view or rear view of the imaged anatomy orientation during diagnosis.

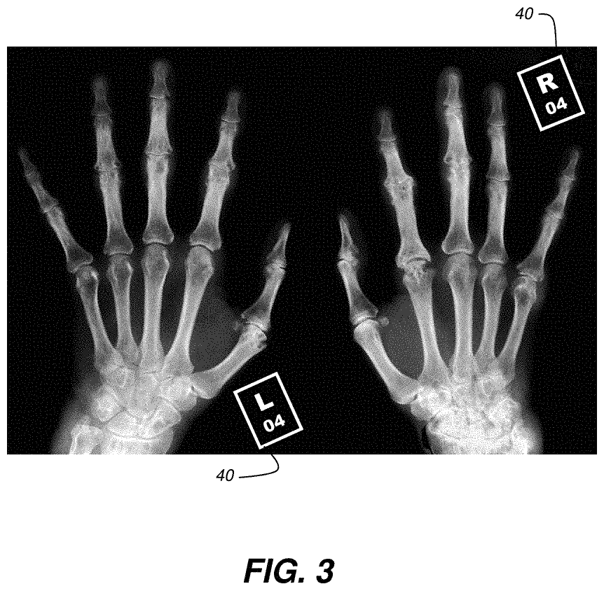

FIG. 3 shows a plan view having multiple anatomical markers 40 with additional information for an x-ray that shows both hands of a patient. Markers 40 can also include identifying information for use by the technologist, practitioner, or site. Because markers 40 are added as part of image post-processing in digital imaging workflow, it is possible to provide various information as desired on the acquired image data.

Marker 40 information content and placement preferences can vary depending on the anatomy that is imaged as well as on particular practices or requirements of the imaging facility or of individual technologists or practitioners.

Marker positions and orientation on the acquired image may conform to site-specific practices, imposing requirements for fixed content fields and positions in designing the associated software for image markup and presentation. The marker can be stored in a preferred position as a layer or overlay with selectable visibility, or as a part of the image content that is always visible.

Given these considerations and the overall goal of improving operator workflow, efficiency, and accuracy, an embodiment of the present disclosure provides automatic positioning logic that performs sufficient scene analysis for determining a suitable position for each marker and provides an initial marker placement for operator approval or confirmation. The automatic positioning logic detects one of a set of established patterns for scene content in the generated x-ray image and identifies an appropriate location on the image for marker placement for the identified pattern. Automatic positioning logic can employ any of a number of types of pattern detection, including algorithmic pattern detection processing or machine learning, for example.

Using various utilities for aspects of scene analysis, operator preference, and other variable data, the radiography system 100 can be configured to automate marker annotation or configuration and positioning for each type of acquired radiographic image that is generated at a facility. This aspect of system operation can help to free the technologist from repetitive tasks and tedious decision making, as well as to help provide x-ray images in a standardized format that simplifies image content interpretation and conforms to requirements or preferences of different practitioners.

### Processing Workflow

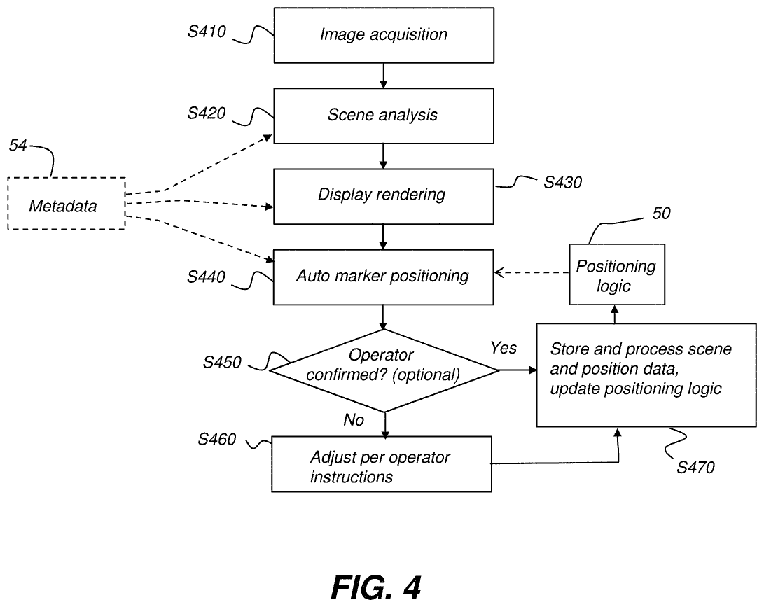

The workflow sequence given in FIG. 4 shows a procedure for automating marker placement on the acquired image, according to an embodiment of the present disclosure. A radiographic image is acquired at digital radiography system 100 (FIG. 1) in an image acquisition step S410. In addition to the image content itself, additional metadata is optionally obtained that can include information such as an operator/technologist/practitioner ID, patient identification, image type, date/time of acquisition, and other data. A scene analysis step S420 classifies or characterizes the image to identify type (such as chest x-ray, extremity type, etc.), view (AP or PA for chest type, one or both hands, left or right knee, etc.), patient position (supine, erect), and other aspects of the imaged content.

Scene analysis step S420 can generate information that thereafter accompanies the acquired image as metadata as well as performing pattern recognition functions useful for determining anatomical orientation for marker placement. The scene analysis can be automated, such as using image analysis software for segmentation or for pattern recognition, including machine learning software that operates by conditioning a neural network according to a training set containing a large number of example x-ray images.

Along with scene analysis procedures, the image content is displayed on a monitor in a display rendering step S430. Steps S420 and S430 can be executed simultaneously, or in any suitable order.

Continuing with the FIG. 4 workflow, an automatic marker positioning step S440 superimposes one or more digital markers 40 onto the displayed scene content. Step S440 employs positioning logic 50 for determining radiography system 100 assigned placement, orientation, and content of anatomical markers 40 without operator intervention. As described in more detail subsequently, positioning logic 50 can use combined metadata that is associated with the acquired image as well as image content, along with established or predetermined rules of practice that guide marker 40 positioning under various conditions.

The workflow process of FIG. 4 includes an optional check step S450 that displays the initial system-assigned anatomical marker 40 position identified by system logic to allow operator confirmation of marker 40 positioning or, alternately, accepts operator offset or other adjustment of marker 40 position, orientation, or content in an adjustment step S460. For step S460, anatomical marker 40 position can be highlighted, such as using a color outline or other on-screen treatment that attracts attention to the given marker 40 location at the initial system-assigned position.

Operator input for spatial repositioning of marker 40 or for front/rear view adjustment can include touch screen entry or use of other visual pointers for moving a displayed and highlighted anatomical marker 40 into a preferred position. Acceptance of the displayed position as the preferred or operator-confirmed anatomical orientation marker position can be verified by operator entry of an on-screen or keyboard command Once accepted, the confirmed anatomical marker appearance may be changed to remove highlighting, for example. The results of operator confirmation or adjustment can then be stored as part of the image content in an update step S470.

According to an embodiment, metadata 54 can be used to identify the location of anatomy of interest in the acquired image data. Operator adjustment in step S460 is checked and a warning message displays if the operator moves the marker position within the identified anatomy of interest.

Storage of the combined subject anatomy image and marker content can be in standard DICOM (Digital Imaging and Communications in Medicine) format, for example. Where machine-learning logic is utilized, positioning logic 50 can then add updated information on marker 40 positioning based on the most recent processing of marker content.

### Positioning Logic 50 Input and Performance Considerations

As noted previously, positioning logic 50 can obtain various information from the acquired image content itself as well as accessing related information from metadata concerning the acquired image in order to determine appropriate marker content and placement on the image. Information useful as input for positioning logic 50 can include the following:

(i) image type. Scene analysis results from step S420 or from metadata 54 generated and associated with the image can include information that classifies the type of x-ray (for example, chest PA, chest AP, left knee, right elbow). Image type information can then be used to determine how many markers are deployed as well as preferred positioning of markers within the image and their front-view or rear-view orientation. For a standard chest image, for example, it may be sufficient to employ a single marker indicating left (L) (FIG. 2) or right (R) side of the patient. Patient orientation (for example, erect or supine) may also be relevant to marker placement.
(ii) Anatomy of interest or region of interest (ROI). Within the scene content of the image, a region of interest (ROI) can be defined according to the anatomy that is the subject of clinical/diagnostic interest. For an image of the chest area, for example, the ROI can be a portion of the rib cage, heart, the lung fields, or other anatomy features.
(iii) Image orientation. The image orientation with respect to the patient anatomy can be a determining factor for marker positioning. For example, for a rear-view rendering of image content, standard practice at many sites is to reverse the marker so that a left (L) or right (R) designation appears to be reversed on the display screen.
(iv) Clipping or other image anomaly. Depending on factors such as patient size, relative detector positioning, and anatomy of interest, the subject anatomy can be partially clipped and thus portions of the anatomy are omitted from the image area, or the subject outline may lie over areas of the image that are normally free for marker placement.
(v) Technologist or practitioner preferences. The positioning logic 50 can further identify the technologist or practitioner and may apply standard practices based on training and on the given practitioner identification.

Because it must balance numerous variable factors and act based on information that can vary significantly case-by-case, positioning logic 50 must perform more than straightforward pattern-matching functions and in many cases can execute complex functions that can emulate the judgment and decision-making processes of a trained radiography technologist related to anatomical orientation marker positioning.

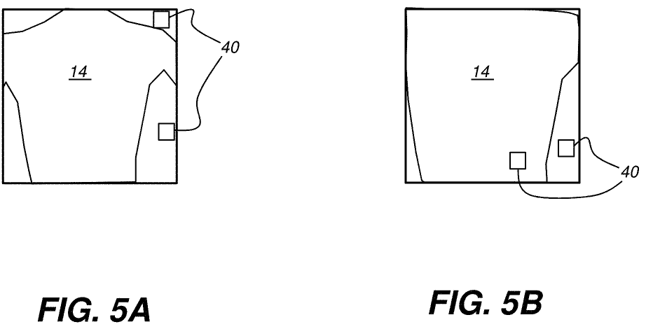

An aspect of positioning logic 50 relates to pattern matching, identifying image content according to standard imaging patterns established by anatomical relationships. For many x-ray images of a particular type, pattern matching can be sufficient for identifying anatomy ROI boundaries and ascertaining that particular areas of the acquired x-ray image are suitable for marker 40 placement. As shown schematically in FIG. 5A, there can be standard positions within a chest x-ray image and outside of the anatomy area that are readily usable for marker 40 positioning in many cases. However, as shown schematically in FIG. 5B, there can be chest x-ray images for which standard marker 40 placement is not suitable. This can be due to the size of the patient 14 or due to less-than-desirable positioning of the detector relative to the patient. In some cases, for example, marker placement within the anatomy, but at a position located away from the anatomy of interest or outside the boundaries of the region of interest (ROI) can be the best solution available. For a chest x-ray image, as shown in FIG. 5B, for example, marker 40 placement within the area of the abdomen may be an acceptable alternative where there is no other suitable position outside the imaged anatomy boundaries.

There may also be cases wherein the anatomy of interest dictates whether or not a particular marker position is acceptable. For imaging a clavicle, for example, rules for marker position may be relaxed over restrictions when imaging lung fields.

### Training for Positioning Logic 50

In general, many types of interactive and interpretive applications typically have non-trivial solutions and deal with large amounts of data, often of different types or dimension, obtained for different end-uses and from different sources. For example, the computational task of identifying suitable positions for orientation markers on a particular x-ray image can involve pattern recognition and analysis of 2-D image data as well as analysis of metadata that is associated with the image data. For example, x-rays of the chest area taken for assessment of lung conditions or for clavicle fracture necessarily both include portions of the same anatomy, but have different regions of interest (ROI) and may be obtained using different x-ray technique settings. Anatomy orientation marker considerations for these two different image types may overlap somewhat, but do not coincide; different image content could be obscured for either image type without compromising diagnostic utility. In such situations, machine learning techniques can offer useful solutions for proper identification and classification of image content as well as for superimposing marker content in appropriate positions. Machine learning, in the context of an embodiment of the present disclosure, can generate the logic for forming a neural network that can be applied for the complex pattern recognition and decision-making logic used for suitable anatomical marker placement.

As noted previously, there can be a number of factors that affect marker 40 positioning for the acquired x-ray, including both image content and metadata associated with the image, with region of interest (ROI) content, and with the patient condition. An embodiment of the present disclosure can employ machine learning for rapid pattern recognition and response in marker 40 position assignment. The logic flow diagram of FIG. 4 can be adapted and applied as a training sequence for generating and training positioning logic 50, along with ongoing improvement from day-to-day use that refines the positioning logic.

According to an aspect of the present disclosure, one or more radiography workstation components may perform the training phase. The methods by which training can be done include, but are not limited to, Support Vector Machines, Neural Networks, Decision Trees, naive Bayes, Logistic Regression, and other techniques from supervised, semi-supervised, and unsupervised learning. A training or “model-derivation” aspect of the disclosed method may be practiced with any available training techniques that yield a method for classifying. Once the training is complete, a number of models can be derived for different image types. The workstation component can automatically create a sequence that selects among the derived models for individual images.

When used for training, metadata 54 related to the image scene content, such as data identifying the image type and patient condition, for example, can be particularly useful for classification and for scene analysis step S420. As described previously, scene analysis step S420 provides information for image characterization that is particularly useful for initial marker positioning.

The training sequence particularly relies on update step S470 for updating and improving the positioning logic 50 based on ongoing results from image-to-image processing and marker 40 placement adjustment. With this procedure, the training process can be continually refined, allowing further customization to meet the particular needs of a facility or the specific requirements of a practitioner.

Utilities and techniques for obtaining the recognition and response functions needed for machine learning and for forming positioning logic 50 using neural network logic utilities are well known to those skilled in the programming arts. Logic for classifying image type, for analyzing image content and related data to determine image orientation and to identify one or more region(s) of interest, and for identifying a system-assigned position and orientation of a digital marker can be generated using appropriate training software applied to case-by-case examples from a training database. In an iterative training process, a skilled observer can position anatomical orientation markers on each image from the training database, with results recorded and used to condition the neural network data structures. As training proceeds, relative probability weightings that relate to network logic are continually assigned and adjusted with successive example cases. After a sufficient number of images have been processed, the relative weightings related to decision-making probabilities tend to stabilize, changing very little with each subsequent case and providing neural network response that identifies image content and recommended marker positioning with close approximation to results achieved by a skilled observer. According to an embodiment of the present disclosure, training can be considered complete when system response is not perceptibly changed, with little or no change to the neural network positioning logic 50 results after a given number of iterations.

As will be appreciated by one skilled in the art, aspects of the present invention may be embodied as a system, method, or computer program product. Accordingly, aspects of the present invention may take the form of an entirely hardware embodiment, an entirely software embodiment (including firmware, resident software, micro-code, etc.), or an embodiment combining software and hardware aspects that may all generally be referred to herein as a “service,” “circuit,” “circuitry,” “module,” and/or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Program code and/or executable instructions embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer (device), partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described herein with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method of operating a digital radiographic imaging system, the method comprising:
capturing a digital radiographic image of a subject anatomy;
automatically placing within the captured digital radiographic image a digital anatomical marker indicative of a spatial orientation of the subject anatomy;
displaying the captured radiographic image having the digital anatomical marker to an operator, including the operator confirming a position of the automatically placed digital anatomical marker in the captured radiographic image by repositioning the marker or not repositioning the marker in the captured radiographic image; and
storing the captured radiographic image having the digital anatomical marker positioned at the operator confirmed position thereof.

2. The method of claim 1, further comprising classifying the captured radiographic image using pattern recognition.

3. The method of claim 2, further comprising identifying a position to place the digital anatomical marker using a neural network module.

4. The method of claim 3, further comprising updating the neural network module according to the operator confirmed position thereof.

5. The method of claim 3, further comprising training the neural network module to identify the position to place the digital anatomical marker assign using a plurality of digital radiographic images having operator confirmed placements of digital anatomical markers.

6. The method of claim 1, further comprising an operator defining an alternative position of the digital anatomical marker and storing the operator defined alternative position.

7. The method of claim 1, further comprising the anatomical marker indicating a left or right side of the subject anatomy.

8. A method for processing a digital radiographic image of a patient anatomy, the method comprising:
acquiring a digital radiographic image of the patient anatomy;
automatically superimposing an anatomical marker at a first position within the digital radiographic image of the patient anatomy, including applying neural network logic trained to determine a position of the anatomical marker according to a plurality of digital radiographic images having anatomical markers placed therein;
displaying the digital radiographic image of the patient anatomy and the superimposed anatomical marker, the anatomical marker indicating at least a left side or right side orientation of the patient anatomy within the displayed digital radiographic image thereof; and
a viewer of the displayed digital radiographic image of the patient indicating a preferred position of the anatomical marker by electronically storing the digital radiographic image of the patient anatomy having the anatomical marker at the first position or by repositioning the anatomical marker to a different position than the first position and electronically storing the digital radiographic image of the patient anatomy having the repositioned anatomical marker.

9. The method of claim 8, further comprising identifying a region of interest in the acquired digital radiographic image of the patient anatomy.

10. The method of claim 9, wherein the first position of the anatomical marker lies outside the identified region of interest.

11. The method of claim 8, further comprising applying the neural network logic to define an anatomy of interest within the acquired digital radiographic image of the patient anatomy and determining whether the anatomical marker at the first position or at the different position than the first position lies within the defined anatomy of interest.

12. The method of claim 8, further comprising highlighting the anatomical marker and displaying the digital radiographic image of the patient anatomy having the highlighted anatomical marker.

13. The method of claim 8, further comprising changing an appearance of the stored repositioned anatomical marker after the viewer stores the digital radiographic image of the patient anatomy having the anatomical marker at the first position.

14. The method of claim 8, further comprising transmitting over a network the acquired digital radiographic image of the patient anatomy having the preferred position of the anatomical marker.

15. A method for processing a digital radiographic (DR) image executed at least in part by a computer, the method comprising:
acquiring a digital radiographic image of a patient anatomy including associated metadata that indicates a region of interest (ROI) within the patient anatomy;
displaying the digital radiographic image of the patient anatomy and an anatomical marker superimposed by the computer onto the digital radiographic image at a computer determined first position outside the ROI, wherein the anatomical marker indicates at least a left or right side of the patient anatomy;
accepting an operator instruction to move the anatomical marker from the first position to a second position offset from the first position; and
storing the digital radiographic image of the patient anatomy having the anatomical marker at the second position.

16. The method of claim 15, further comprising applying a machine-learned algorithm to the acquired digital radiographic image of the patient anatomy.

17. The method of claim 15, further comprising training the machine-learned algorithm using a plurality of stored radiographic images of patient anatomies each having an anatomical marker positioned therein by a human.

18. The method of claim 17, further comprising identifying boundaries of the ROI and training the machine-learned algorithm according to the second position of the anatomical marker.

19. The method of claim 15, wherein acquiring the digital radiographic image of the patient anatomy comprises acquiring scanned data from a computed radiography system.
